(19) United States
(12) Patent Application Publication
(10) Pub. No.: US 2023/0145764 A1
(43) Pub. Date: May 11, 2023

(54) BLOOD-BASED TUMOR MUTATION BURDEN PREDICTS OVERALL SURVIVAL IN NSCLC

(71) Applicant: MEDIMMUNE, LLC, Gaithersburg, MD (US)

(72) Inventors: Koustubh RANADE, GAITHERSBURG, MD (US); Brandon W. HIGGS, GAITHERSBURG, MD (US); Rajiv G. RAJA, GAITHERSBURG, MD (US); Philip Z. BROHAWN, GAITHERSBURG, MD (US); Han SI, GAITHERSBURG, MD (US); Michael A. KUZIORA, GAITHERSBURG, MD (US)

(21) Appl. No.: 17/961,827

(22) Filed: Oct. 7, 2022

Related U.S. Application Data
(63) Continuation of application No. 16/710,938, filed on Dec. 11, 2019.
(60) Provisional application No. 62/889,199, filed on Aug. 20, 2019.
(60) Provisional application No. 62/778,667, filed on Dec. 12, 2018.

(51) Int. Cl. C12Q 1/6886; A61K 39/395; A61P 35/00

(52) U.S. Cl. CPC C12Q 1/6886; A61K 39/3955; A61P 35/00; C12Q 2600/156; C12Q 2600/106

(57) ABSTRACT

The disclosure generally relates to methods for treating non-small cell lung cancer patients based on use of blood-based tumor mutation burden to predict overall survival in patients treated with durvalumab, tremelimumab, and/or a chemotherapy agent. The disclosure also relates to methods for treating non-small cell lung cancer patients based on identification of mutations in circulating tumor DNA associated with sensitivity or resistance to immunotherapy.

This application is a continuing application of U.S. application Ser. No. 16/710,938, filed on Dec. 11, 2019, which claims the benefit of both U.S. Provisional Application No. 62/889,199, filed on Aug. 20, 2019, and U.S. Provisional Application No. 62/778,667, filed on Dec. 12, 2018, the disclosures of each of which are incorporated by reference herein in their entirety.

### FIELD OF THE DISCLOSURE

The present disclosure generally relates to methods for treating non-small cell lung cancer patients based on use of blood-based tumor mutation burden to predict overall survival in patients treated with durvalumab and/or tremelimumab, and/or a chemotherapy agent. The disclosure also relates to methods for treating non-small cell lung cancer patients based on identification of mutations in circulating tumor DNA associated with sensitivity or resistance to immunotherapy.

### BACKGROUND OF THE DISCLOSURE

Non-small cell lung cancer (“NSCLC”) patients with high pretreatment tumor mutational burden (“TMB”) have demonstrated improved outcomes after treatment with immune checkpoint inhibitors (Yarchoan et al., N. Engl. J. Med. 377(25): 2500-01 (2017); Snyder et al., N. Engl. J. Med. 371(23): 2189-99 (2014); Le et al., Science 357(6349): 409-13 (2017); Rizvi et al., Science 348(6230): 124-28 (2015); Rizvi et al., J. Clin. Oncol. 36(7): 633-41 (2018); Hellmann et al., Cancer Cell 33(5): 843-52 (2018); Carbone et al., N. Engl. J. Med. 376(25): 2415-26 (2017); Hellmann et al., N. Engl. J. Med. 378(22): 2093-104 (2018)). TMB measured in the blood has also emerged as a promising new approach to enrich for NSCLC patients responding to PD-1/L1 therapy (Gandara et al., Ann. Oncol. 28 (Suppl 5): v460-v496 (2017); Planchard et al., Ann. Oncol. 29 (Suppl 4): iv192-iv237 (2018)). Reports have shown that NSCLC patients in both first- and second-line settings with high blood TMB (“bTMB”) had improved progression-free survival and response rates. However, a correlation between either tissue TMB (“tTMB”) or bTMB with overall survival in NSCLC patients treated with an anti-PD-1/L1 antibody has not been shown.

### SUMMARY OF THE DISCLOSURE

The disclosure provides a method of predicting success of cancer treatment in a patient in need thereof, comprising determining the patient's tumor mutational burden (TMB), wherein a high TMB predicts success of treatment.

The disclosure also provides a method of treating cancer in a patient in need thereof, comprising: (a) determining the patient's TMB; (b) determining whether the TMB is high or low; and (c) treating or continuing treatment if TMB is high or not treating or discontinuing treatment if TMB is low.

The disclosure further provides a method of predicting success of cancer treatment in a patient in need thereof, comprising determining if the patient has a somatic mutation in AT-rich interactive domain-containing protein 1A gene (ARID1A), wherein a somatic mutation predicts success of treatment.

The disclosure further provides a method of treating cancer in a patient in need thereof, comprising: (a) determining whether the patient has a somatic mutation in at least one of serine/threonine kinase 11 gene (STK11), Kelch-like ECH-associated protein 1 gene (KEAP1), AT-rich interactive domain-containing protein 1A gene (ARID1A), or K-Ras gene; and (b) treating or continuing treatment if patient has a somatic mutation in at least one of serine/threonine kinase 11 gene (STK11), Kelch-like ECH-associated protein 1 gene (KEAP1), AT-rich interactive domain-containing protein lA gene (ARID1A), or K-Ras gene.

Other features and advantages of the disclosure will be apparent from the detailed description, and the claims.

### BRIEF DESCRIPTION OF THE DRAWINGS/FIGURES

FIG. 1 shows the list of genes included in the TMB analysis.

FIG. 2 shows overall survival in patients with PD-L1 expression on tumor cells (TC) ≥25% treated with durvalumab (D) versus chemotherapy (CT) or durvalumab and tremelimumab (D+T) versus chemotherapy (CT).

FIG. 3 shows progression free survival (PFS) in patients with PD-L1 expression on tumor cells (TC) ≥25% treated with durvalumab versus chemotherapy or durvalumab and tremelimumab versus chemotherapy.

FIG. 4 shows a primary analysis population. The analysis was performed using a Cox proportional hazards model with a term for treatment and the subgroup covariate of interest. Subgroups were according to sex, age, immune cell PD-L1 expression, histology, smoking history, and race. The analysis of subgroups according to performance status was post hoc. *97.54% CI is shown.

FIG. 5 shows correlation of two TMB measurement tools in the MYSTIC study. The correlation plot is based on 352 patients with matched blood and tissue TMB data. The reference line is estimated using linear regression.

FIGS. 6A-6C show overall survival in the ITT and blood and tissue TMB evaluable populations.

FIG. 7 shows analysis of overall survival across blood TMB cut-offs.

FIG. 8 shows overall survival rates in patient with blood TMB ≥16 and <16 mut/Mb.

FIG. 9 shows a Venn diagram indicating overlap of patient subgroups based on blood TMB and PD-L1. *Percentages are calculated from the intention-to-treat population (all randomized patients; N=1118).

FIG. 10 shows overall survival rates in patient with blood TMB ≥20 and <20 mut/Mb.

FIG. 11 shows progression free survival (PFS) in patient with blood TMB ≥20 and <20 mut/Mb.

FIGS. 12A-12B shows overall survival rates in patient with blood TMB ≥10 and <10 mut/Mb.

FIG. 13 shows the TMB Algorithm.

FIG. 14 shows overall survival (OS) in patients with PD-L1 expression on tumor cells (TC) ≥50%) treated with durvalumab and tremelimumab (D+T) versus chemotherapy (CT).

FIG. 15 shows overall survival (OS) in patients with PD-L1 expression on tumor cells (TC) ≥1%) treated with durvalumab and tremelimumab (D+T) versus chemotherapy (CT).

FIG. 16 shows that combining bTMB high or tumor cells (TC) <1% improves prevalence but reduces efficacy.

FIG. 17 shows that combining bTMB high or tumor cells (TC) ≥25% improves prevalence but reduces efficacy.

FIG. 18 shows the prevalence of mutations in the genes KEAP1, STK11, and ARIDIA in patients in MYSTIC study. 324 (of 943 evaluable) patients had mutations in one of the 3 genes KEAP1, STK11, or ARID1A.

FIG. 19 shows prevalence of mutations according to histology and treatment. STK11 and KEAP1 mutations were more prevalent in patients with nonsquamous histology compared with squamous histology. STK11, KEAP1, and ARID1A mutation prevalence was balanced between treatment arms.

FIG. 20 shows prevalence of mutations according to bTMB status.

FIG. 21 shows prevalence of mutations according to PD-L1 expression.

FIG. 22 shows objective response rates for treatment with durvalumab and tremelimumab (durvalumab+tremelimumab), durvalumab monotherapy (durvalumab), or chemotherapy according to mutation status in patients.

FIG. 23 shows overall survival for KEAP1m vs KEAP1wt in all mutation-evaluable patients treated with durvalumab and tremelimumab, durvalumab monotherapy, or chemotherapy. Patients treated with durvalumab and tremelimumab, durvalumab monotherapy, or chemotherapy were included in each group; m=mutation-positive; mOS=median overall survival; wt=wild type.

FIG. 24 shows overall survival for KEAP1m vs KEAP1wt in patients treated with durvalumab monotherapy versus chemotherapy or durvalumab+tremelimumab versus chemotherapy.

FIG. 25 shows overall survival for STK11m vs STK11wt in all mutation-evaluable patients. Patients treated with durvalumab and tremelimumab, durvalumab monotherapy, or chemotherapy were included in each group.

FIG. 26 shows overall survival for STK11m vs STK11wt in patients treated with durvalumab monotherapy versus chemotherapy or durvalumab+tremelimumab versus chemotherapy.

FIG. 27 shows overall survival for STK11m/KEAP1m and STK11m/KRASm versus wild type in all mutation-evaluable patients. Patients treated with durvalumab and tremelimumab, durvalumab monotherapy, or chemotherapy were included in each group.

FIG. 28 shows overall survival for ARID1Am and ARID1Awt in all mutation-evaluable patients. Patients treated with durvalumab and tremelimumab, durvalumab monotherapy, or chemotherapy were included in each group.

FIG. 29 shows overall survival for ARID1Am vs ARID1wt in patients treated with durvalumab monotherapy versus chemotherapy or durvalumab+tremelimumab versus chemotherapy.

### DETAILED DESCRIPTION OF THE DISCLOSURE

The present disclosure generally relates to methods for treating non-small cell lung cancer patients based on use of blood-based tumor mutation burden to predict overall survival in patients treated with durvalumab and/or tremelimumab, and/or a chemotherapy agent. The disclosure also relates to methods for treating non-small cell lung cancer patients based on identification of mutations in circulating tumor DNA (ctDNA) associated with sensitivity or resistance to immunotherapy.

The disclosure is based, at least in part, on the identification of unique patient subsets through bTMB. As described herein, bTMB was more predictive of overall survival than levels of PD-L1 expression for durvalumab treatment in combination with tremelimumab. In some embodiments, bTMB is also more predictive of overall survival than levels of PD-L1 expression for durvalumab monotherapy treatment+/−a chemotherapy agent. In further embodiments, bTMB is more predictive of overall survival than levels of PD-L1 expression for durvalumab treatment in combination with tremelimumab+/−a chemotherapy agent.

As utilized in accordance with the present disclosure, unless otherwise indicated, all technical and scientific terms shall be understood to have the same meaning as commonly understood by one of ordinary skill in the art. Unless otherwise required by context, singular terms shall include pluralities and plural terms shall include the singular.

In some embodiments provided herein is a method of predicting success of cancer treatment in a patient in need thereof, comprising determining the patient's tumor mutational burden (TMB), wherein a high TMB predicts success of treatment.

“Tumor mutational burden” or “TMB” refers to the quantity of mutations found in a tumor. TMB varies among different tumor types. Some tumor types have a higher rate of mutation than others. TMB can be measured by a variety of tools known in the field. In certain embodiments, these tools are the Foundation Medicine and Guardant Health measurement tools. As described herein, evaluation of potential neoantigen encoding mutations in a particular set of genes was found to be correlative for treatment with durvalumab in combination with tremelimumab. Determining whether a tumor has high or low levels of tumor mutational burden can be determined by comparison to a reference population having similar tumors and determining median or mean level of expression. In some embodiments, a high TMB is defined as ≥12 to ≥20 mutations/megabase (mut/Mb). In other embodiments, a high TMB is defined as ≥16 mutations/megabase (mut/Mb). In other embodiments, a high TMB is defined as ≥20 mutations/megabase (mut/Mb).

As used herein the term “MYSTIC” refers to Study NCT02453282, which is a phase III open label first line therapy study of durvalumab, with or without tremelimumab, versus standard of care in NSCLC.

In some embodiments, the method comprises treatment with durvalumab. The term “durvalumab” as used herein refers to an antibody that selectively binds PD-L1 and blocks the binding of PD-L1 to the PD-1 and CD80 receptors, as disclosed in U.S. Pat. No. 9,493,565 (referred to as “2.14H9OPT”), which is incorporated by reference herein in its entirety. The fragment crystallizable (Fc) domain of durvalumab contains a triple mutation in the constant domain of the IgG1 heavy chain that reduces binding to the complement component C1q and the Fcγ receptors responsible for mediating antibody-dependent cell-mediated cytotoxicity (“ADCC”). Durvalumab can relieve PD-L1-mediated suppression of human T-cell activation in vitro and inhibits tumor growth in a xenograft model via a T-cell dependent mechanism.

In some embodiments, the methods disclosed herein comprise treatment with tremelimumab. The term “tremelimumab” as used herein refers to an antibody that selectively binds a CTLA-4 polypeptide, as disclosed in U.S. Pat. No. 8,491,895 (referred to as “clone 11.2.1”), which is incorporated by reference herein in its entirety. Tremelimumab is specific for human CTLA-4, with no cross-reactivity to related human proteins. Tremelimumab blocks the inhibitory effect of CTLA-4, and therefore enhances T-cell activation. Tremelimumab shows minimal specific binding to Fc receptors, does not induce natural killer (NK) ADCC activity, and does not deliver inhibitory signals following plate-bound aggregation.

In some embodiments, the methods disclosed herein comprise treatment with a chemotherapy agent comprising at least one of abraxane, carboplatin, gemcitabine, cisplatin, pemetrexed, or paclitaxel. In some embodiments, the chemotherapy agent comprises abraxane+carboplatin, gemcitabine+cisplatin, gemcitabine+carboplatin, pemetrexed+carboplatin, pemetrexed+cisplatin, or paclitaxel+carboplatin.

In some embodiments, the methods disclosed herein comprise treatment with durvalumab, tremelimumab, and a chemotherapy agent. In other embodiments, the methods disclosed herein comprise treatment with durvalumab and a chemotherapy agent. In other embodiments, the methods disclosed herein comprise treatment with durvalumab.

In some embodiments, the patient has a somatic mutation in at least one of serine/threonine kinase 11 gene (STK11), Kelch-like ECH-associated protein 1 gene (KEAP1), AT-rich interactive domain-containing protein 1A gene (ARID1A), or K-Ras gene. STK11 and KEAP1 mutation status was prognostic for OS in patients with metastatic non-small cell lung cancer (mNSCLC). In some embodiments, mutations in STK11 or KEAP1 mNSCLC are prognostic for shorter OS as compared to patients with wildtype STK11 or KEAP1 mNSCLC. In some embodiments, mutations in ARID1A are used as a biomarker predictive of improved OS in patients receiving durvalumab+tremelimumab treatment.

The term “ARID1A” encompasses “full-length” unprocessed ARID1A as well as any form of ARID1A that results from processing in the cell. The term also encompasses naturally occurring variants of ARID1A, e.g., splice variants or allelic variants.

The term “KEAP1” encompasses “full-length” unprocessed KEAP1 as well as any form of KEAP1 that results from processing in the cell. The term also encompasses naturally occurring variants of KEAP1, e.g., splice variants or allelic variants.

The term “STK11” encompasses “full-length” unprocessed STK11 as well as any form of STK11 that results from processing in the cell. The term also encompasses naturally occurring variants of STK11, e.g., splice variants or allelic variants.

The term“K-Ras” encompasses “full-length” unprocessed K-Ras as well as any form of K-Ras that results from processing in the cell. The term also encompasses naturally occurring variants of K-Ras, e.g., splice variants or allelic variants.

In some embodiments provided herein is a method of treating cancer in a patient in need thereof, comprising:
(a) determining the patient's TMB;
(b) determining whether the TMB is high or low; and
(c) treating or continuing treatment if TMB is high or not treating or discontinuing treatment if TMB is low.

Determining whether a TMB is high may vary from tumor type to tumor type. Determining whether a tumor has high or low levels of tumor mutational burden can be determined by comparison to a reference population having similar tumors and determining median or mean level of expression. In some embodiments, the levels of TMB are divided as low (1-5 mutations/mb), intermediate (6-15 mutations/mb), and high (≥16 mutations/mb).

In some embodiments, the success of treatment is determined by an increase in OS as compared to standard of care. “Standard of care” (SOC) and “platinum-based chemotherapy” refer to chemotherapy treatment comprising at least one of abraxane, carboplatin, gemcitabine, cisplatin, pemetrexed, or paclitaxel. In some embodiments, the SOC comprises abraxane+carboplatin, gemcitabine+cisplatin, gemcitabine+carboplatin, pemetrexed+carboplatin, pemetrexed+cisplatin, or paclitaxel+carboplatin.

As used herein, Overall Survival (“OS”) relates to the time-period beginning on the date of treatment until death due to any cause. OS may refer to overall survival within a period of time such as, for example, 12 months, 18 months, 24 months, and the like.

In some embodiments, provided herein is a method of predicting success of cancer treatment in a patient in need thereof, comprising determining if the patient has a somatic mutation in AT-rich interactive domain-containing protein 1A gene (ARID1A), wherein a somatic mutation predicts success of treatment.

In some embodiments, provided herein is a method of treating cancer in a patient in need thereof, comprising: (a) determining whether the patient has a somatic mutation in at least one of serine/threonine kinase 11 gene (STK11), Kelch-like ECH-associated protein 1 gene (KEAP1), AT-rich interactive domain-containing protein 1A gene (ARID1A), or K-Ras gene; and (b) treating or continuing treatment if patient has a somatic mutation in at least one of serine/threonine kinase 11 gene (STK11), Kelch-like ECH-associated protein 1 gene (KEAP1), AT-rich interactive domain-containing protein 1A gene (ARID1A), or K-Ras gene.

The term “patient” is intended to include human and non-human animals, particularly mammals.

In some embodiments, the methods disclosed herein relate to treating a subject for a tumor disorder and/or a cancer disorder. In some embodiments, the cancer is selected from melanoma, breast cancer, pancreatic cancer, lung cancer (e.g., non-small cell lung cancer (NSCLC) and small cell lung cancer (SCLC)), hepatocellular carcinoma, cholangiocarcinoma or biliary tract cancer, gastric cancer, oesophagus cancer, head and neck cancer, renal cancer, cervical cancer, colorectal cancer, or urothelial bladder cancer.

The terms “treatment” or “treat” as used herein refer to therapeutic treatment. Those in need of treatment include subjects having cancer. In some embodiments, the methods disclosed herein can be used to treat tumors. In other embodiments, treatment of a tumor includes inhibiting tumor growth, promoting tumor reduction, or both inhibiting tumor growth and promoting tumor reduction.

The terms “administration” or “administering” as used herein refer to providing, contacting, and/or delivering a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, and implants.

The terms “pharmaceutical composition” or “therapeutic composition” as used herein refer to a compound or composition capable of inducing a desired therapeutic effect when properly administered to a subject. In some embodiments, the disclosure provides a pharmaceutical composition comprising a pharmaceutically acceptable carrier and a therapeutically effective amount of at least one antibody of the disclosure.

Without limiting the disclosure, a number of embodiments of the disclosure are described below for purpose of illustration.

### EXAMPLES

The Examples that follow are illustrative of specific embodiments of the disclosure, and various uses thereof. They are set forth for explanatory purposes only and should not be construed as limiting the scope of the invention in any way.

### Example 1

### Durvalumab with or Without Tremelimumab in Metastatic

### Non-Small-Cell Lung Cancer

The MYSTIC study described herein was a phase 3 study which compared durvalumab, with or without tremelimumab, with platinum-based chemotherapy as first-line treatment for metastatic NSCLC.

### Patients

Adult patients with Stage IV NSCLC were eligible provided they had not previously received systemic therapy for advanced/metastatic NSCLC, had Eastern Cooperative Oncology Group performance status 0-1, measurable disease according to Response Evaluation Criteria in Solid Tumors version 1.1 (RECIST v1.1) (Chaft et al., Cancer Res. 78(13 Suppl) (abstr. CT113) (2018)), and known tumor PD-L1 expression status prior to randomization. Patients with sensitizing EGFR mutations or ALK rearrangements, and those with symptomatic, unstable brain metastases were excluded.

### Study Design and Treatment

Patients were randomized in a 1:1:1 ratio, with stratification according to PD-L1 TC ≥25% versus<25% and histology, to receive durvalumab 20 mg/kg every 4 weeks, durvalumab 20 mg/kg plus tremelimumab 1 mg/kg every 4 weeks (up to four doses), or 4-6 cycles of platinum-based doublet chemotherapy. Patients continued treatment until objective disease progression (according to RECIST v1.1), development of an adverse event (AE) that necessitated treatment discontinuation, or withdrawal of consent.

### Endpoints and Assessments

The primary endpoints were Overall Survival (OS; time from randomization to death from any cause) for both immunotherapy arms compared with chemotherapy, and Progression Free Survival (PFS; time from randomization to objective disease progression by blinded independent central review [BICR] or death) for durvalumab plus tremelimumab compared with chemotherapy, all in patients with PD-L1 TC ≥25%. Primary endpoints were to be evaluated in patients with PD-L1 TC ≥25%. Secondary endpoints included PFS for durvalumab versus chemotherapy, objective response rate (ORR) and duration of response (DOR) for both immunotherapy arms compared with chemotherapy (all in patients with PD-L1 TC ≥25%), and safety and tolerability. Investigation of the relationship between biomarkers, including TMB, and clinical outcomes were also determined.

PD-L1 expression was evaluated using multiple cut-offs at a central laboratory using the VENTANA PD-L1 (SP263) immunohistochemistry (IHC) assay (Ventana Medical Systems, Tucson, Ariz., USA) (Rebelatto et al., Diagn. Pathol. 11(1): 95 (2016)). Tumor samples obtained within 3 months prior to screening were permitted. Strong analytical agreement has been demonstrated across the dynamic range between the Dako PD-L1 IHC 22C3 pharmDx and VENTANA PD-L1 (SP263) IHC assays (Hirsch et al., J. Thorac. Oncol. 12(2): 208-22 (2017); Ratcliffe et al., Clin. Cancer Res. 23(14): 3585-91 (2017)).

Tumor response was assessed by BICR using RECIST v1.1, with imaging performed every 6 weeks for the first 48 weeks, then every 8 weeks, until confirmed disease progression. Patients were followed for survival. AEs were graded according to National Cancer Institute Common Terminology Criteria for Adverse Events version 4.03.

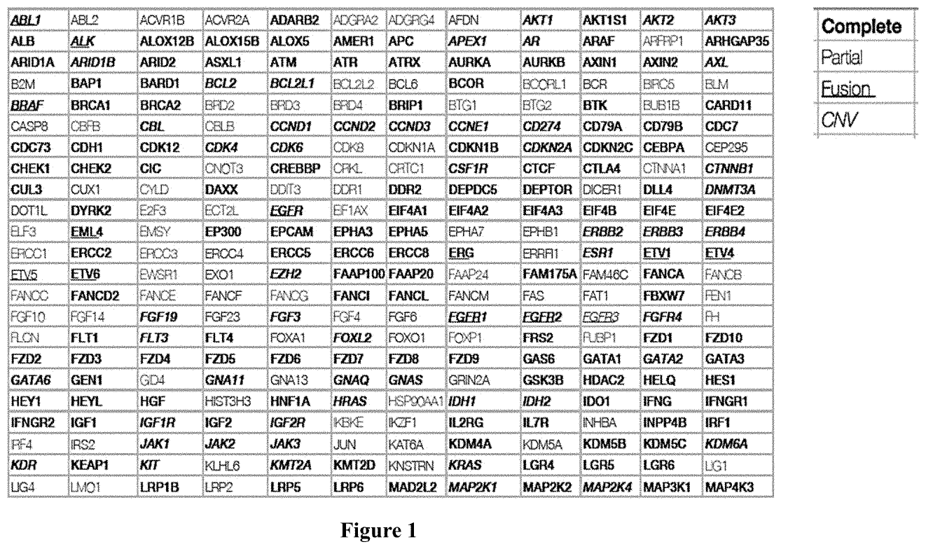

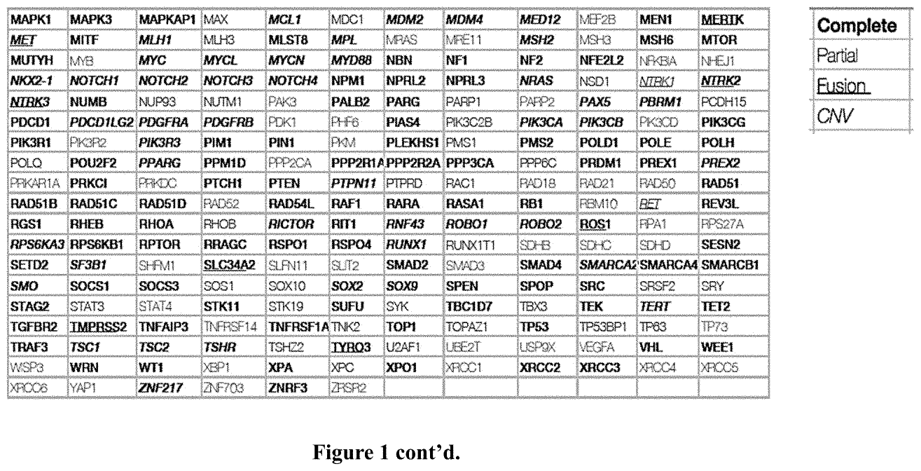

Blood TMB was evaluated using the GuardantOMNI next-generation sequencing platform (Guardant Health, Redwood City, Calif., USA) comprised of 500 genes (FIG. 1). All genes shown in FIG. 1 are potential identifiers of TMB, and the relevance of each gene or combination of genes will vary by patient.

The OMNI TMB algorithm incorporates somatic single nucleotide variants (SNVs), short insertions/deletions (indels), copy number amplifications and fusions, and is optimized to calculate TMB on blood samples with low cell-free circulating tumor DNA content (Merck Sharp & Dohme. Keytruda® (pembrolizumab) Summary of Product Characteristics. Updated March 2019. Available at: https://www.medicines.org.uk/emc/product/6947/smpc (last accessed May 1, 2019); Reck et al., N. Engl. J. Med. 375(19): 1823-33 (2016)). Both synonymous and nonsynonymous SNVs and indels were included, with removal of those with low shedding values, low diversity, and associations with clonal hematopoiesis, germline and oncogenic driver or drug resistance mechanisms. Tissue TMB was evaluated using the FoundationOne tissue next-generation sequencing platform (Foundation Medicine, Cambridge, Mass., USA). The algorithm has been described previously (Merck Sharp & Dohme. Keytruda® (pembrolizumab) prescribing information. Updated April 2019. Available at: https://www.merck.com/product/usa/pi_circulars/k/keytruda/keytruda_pi.pdf (last accessed May 1, 2019)).

### Statistical Analysis

Approximately 1092 patients, including 480 with PD-L1 TC ≥25%, were needed to obtain 231 PFS events for the primary PFS analysis across the durvalumab plus tremelimumab and chemotherapy groups and 225 OS events for the primary OS analysis across each treatment group comparison.

Efficacy was analyzed on an intention-to-treat (ITT) basis, including all randomized patients or subsets of this population based on PD-L1 expression or TMB levels. Safety analyses included all patients who received at least one dose of study treatment (as-treated population).

To control the type I error at 5% (two-sided), a hierarchical multiple testing procedure with gatekeeping strategy was used across endpoints, analysis populations, and treatment regimens. The primary PFS analysis was performed using a stratified log-rank test adjusting for histology, with hazard ratio (HR) and 99.5% confidence interval (CI) estimated using a Cox proportional hazards model. The primary OS analysis was performed using similar methodology, with HRs estimated with two-sided 97.54% and 98.77% Cis for comparisons of durvalumab and durvalumab plus tremelimumab, respectively, with chemotherapy. Survival curves were generated using the Kaplan-Meier method.

For secondary analyses performed on the PD-L1 TC ≥1% and ITT populations, the stratification was additionally adjusted for PD-L1 expression status (TC ≥25% vs. TC <25%). Odds ratios and 95% CI for comparing ORR between treatment groups were calculated using a logistic regression model, adjusted for the same factors as PFS and OS. Prespecified TMB analysis was performed using an unstratified log-rank test, with HRs and 95% Cis estimated using a Cox proportional hazards model.

### Results

Of 1118 randomized patients, 1092 (97.7%) received at least one dose of study treatment (369 received durvalumab, 371 durvalumab plus tremelimumab, and 352 chemotherapy). In the chemotherapy group, the most common regimen was gemcitabine plus carboplatin (49.5%) and pemetrexed plus carboplatin (54.5%) in patients with squamous and nonsquamous histology, respectively. A total of 488 patients had PD-L1 TC ≥25% (primary analysis population; 43.6% of randomized patients). The baseline demographics and disease characteristics of patients with PD-L1 TC ≥25% were generally consistent with the ITT population and balanced between treatment groups.

Among patients with PD-L1 TC ≥25%, 25 in the durvalumab group, 18 in the durvalumab plus tremelimumab group, and 1 in the chemotherapy group remained on study treatment. Of these patients, 5 in the durvalumab group and 1 in the durvalumab plus tremelimumab group were treated through progression, and 5 in the durvalumab plus tremelimumab group received retreatment with tremelimumab. After discontinuation of study treatment, 73 (44.8%) patients in the durvalumab group, 61 (37.4%) in the durvalumab plus tremelimumab group, and 95 (58.6%) in the chemotherapy group received subsequent systemic cancer therapy. Among these patients, immunotherapy was received by 10 (13.7%) of 73 in the durvalumab group, 5 (8.2%) of 61 in the durvalumab plus tremelimumab group, and 64 (67.4%) of 95 in the chemotherapy group.

1. Efficacy

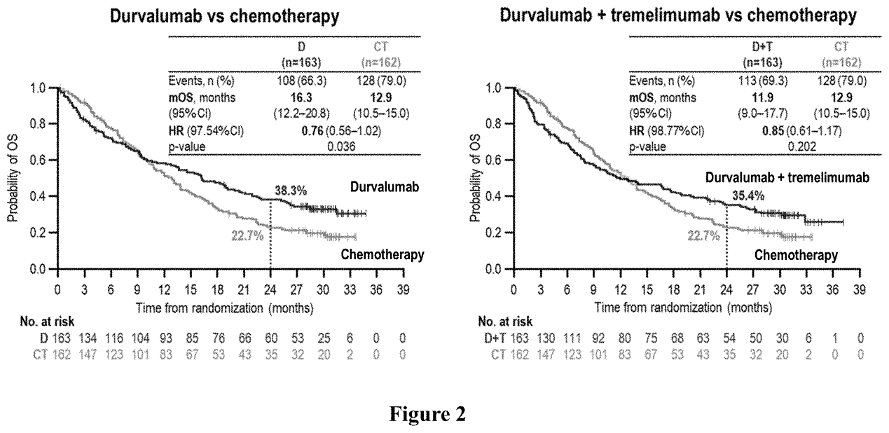

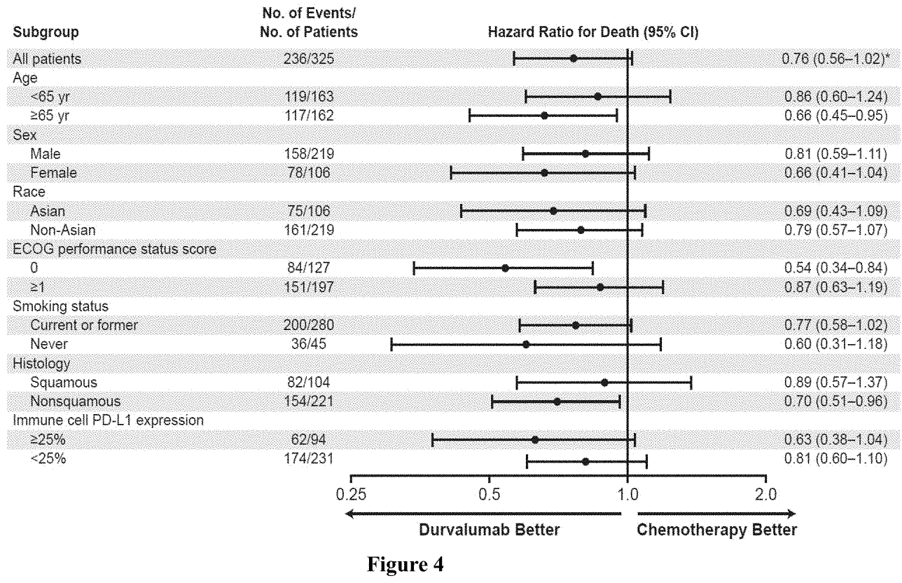

Median follow-up for OS was 30.2 months (range, 0.3-37.2). Durvalumab and durvalumab plus tremelimumab did not statistically significantly improve OS compared with chemotherapy in patients with PD-L1 TC ≥25%. The median OS was 16.3 months with durvalumab versus 12.9 with chemotherapy (HR for death, 0.76; 97.54% CI, 0.56-1.02; P=0.036) (FIG. 2). The 24-month OS rate was 38.3% (95% CI, 30.7-45.7) in the durvalumab group and 22.7% (16.5-29.5) with chemotherapy. Most planned patient subgroups treated with durvalumab had numerical improvement in OS versus chemotherapy (FIG. 4). The median OS was 11.9 months and the 24-month OS rate was 35.4% (95% CI, 28.1-42.8) with durvalumab plus tremelimumab (HR for death vs. chemotherapy, 0.85; 98.77% CI, 0.61-1.17; P=0.202) (FIG. 2). OS in the ITT population and in subgroups defined by different PD-L1 expression levels (TC <1%, ≥1%, ≥25-49%, and ≥50%) is shown in Table 1.

TABLE 1
Overall Survival in the ITT Population and by PD-LI Expression Subgroup.

| | | Durvalumab | | Durvalumab + | | | |
|---|---|---|---|---|---|---|---|
| | | Monotherapy | | Tremelimumab | | Chemotherapy | |
| ITT population* | Number of | 278/374 | | 278/372 | | 297/372 | |
| | events/patients | | | | | | |
| | Median overall | 12.3 | (10.1-14.9) | 11.2 | (9.5-12.9) | 11.8 | (10.5-13.3) |
| | survival, months | | | | | | |
| | (95% CI) | | | | | | |
| | Hazard ratio (95% | 0.96 | (0.81-1.13) | 0.94 | (0.79-1.10)* | — | |
| | CI)† | | | | | | |
| PD-LI TC ≥ 1% | Number of patients | 194/279 | | 221/296 | | 226/289 | |
| | Median overall | 14.6 | (10.5-17.7) | 10.9 | (9.1-13.5) | 12.3 | (10.6-14.6) |
| | survival, months | | | | | | |
| | (95% CI) | | | | | | |
| | Hazard ratio (95% | 0.88 | (0.73-1.07)^{§} | 1.01 | (0.83-1.21)^{‡} | — | |
| | CI)^{†} | | | | | | |
| PD-LI TC ≥ | Number of patients | 33/45 | | 45/55 | | 46/55 | |
| 25-49% | Median overall | 11.1 | (6.2-22.5) | 10.5 | (5.3-16.7) | 13.3 | (8.4-16.3) |
| | survival, months | | | | | | |
| | (95% CI) | | | | | | |
| | Hazard ratio (95% | 0.78 | (0.49-1.23) | 0.95 | (0.62-1.45) | — | |
| | CI)^{t} | | | | | | |
| PD-LI TC ≥ 50% | Number of patients | 75/118 | | 68/108 | | 82/107 | |
| | Median overall | 18.3 | (13.6-22.8) | 15.2 | (8.0-26.5) | 12.7 | (10.3-15.1) |
| | survival, months | | | | | | |
| | (95% CI) | | | | | | |
| | Hazard ratio (95% | 0.76 | (0.55-1.04)8 | 0.77 | (0.56-1.07)8 | — | |
| | CI)† | | | | | | |
| PD-LI TC < 1% | Number of patients | 84/95 | | 57/76 | | 71/83 | |
| | Median overall | 10.1 | (6.7-12.2) | 11.9 | (9.3-18.6) | 10.3 | (7.9-12.9) |

| survival, months | | | | | |
|---|---|---|---|---|---|
| (95% CI) | | | | | |
| Hazard ratio (95% | 1.18 | (0.86-1.62)8 | 0.73 | (0.51-1.04)8 | — |

CI)†

*The ITT population includes all randomizec patients.
^{†}Hazard ratio for death compared with chemotherapy.
^{‡}Secondary endpoint. SPrespe cified subgroup at lalysis. CI, confidence interval; ITT, intendon-to-treat; PD-L1, programmed cell death ligand-1 ; TC, tumor cell.

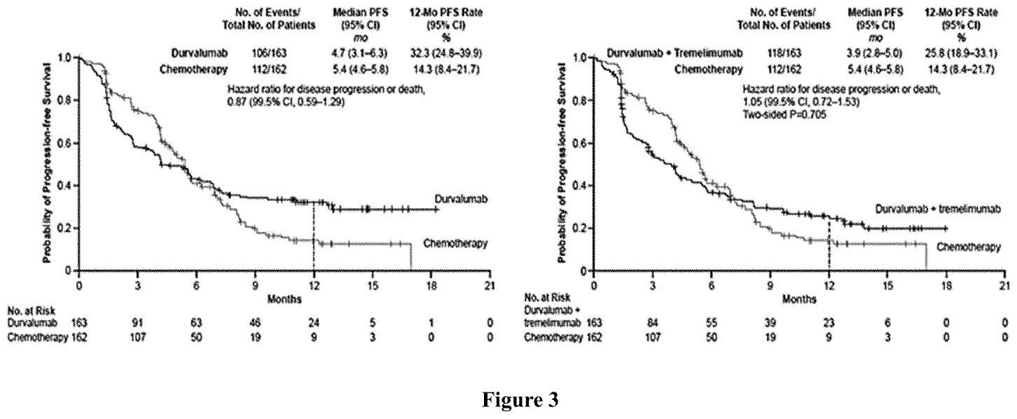

Median follow-up for PFS was 10.6 months (range, 0-18). There was no statistically significant difference in PFS between the durvalumab and chemotherapy groups (secondary endpoint; FIG. 3) or between durvalumab plus tremelimumab and chemotherapy groups (primary endpoint; FIG. 3). Median PFS was 3.9 months (95% CI, 2.8-5.0) with durvalumab plus tremelimumab versus 5.4 (4.6-5.8) with chemotherapy (HR for disease progression or death, 1.05; 99.5% CI, 0.72-1.53; P=0.705); the 12-month PFS rate was 25.8% (95% CI, 18.9-33.1) with durvalumab plus tremelimumab versus 14.3% (8.4-21.7) with chemotherapy.

ORR among patients with PD-L1 TC ≥25% was 35.6% in the durvalumab group, 34.4% in the durvalumab plus tremelimumab group, and 37.7% in the chemotherapy group (Table 2). The median DOR was not reached in the immunotherapy arms versus 4.4 months with chemotherapy. More patients remained in response at 12 months in the immunotherapy treatment groups (61.3%, 54.9%, and 18.0% in the durvalumab, durvalumab plus tremelimumab, and chemotherapy arms, respectively) (Table 2).

TABLE 2
Summary of Tumor Response among Patients with PD-L1 TC ≥ 25%.

| | Durvalumab | | Durvalumab plus | | | |
|---|---|---|---|---|---|---|
| | Monotherapy | | Tremelimumab | | Chemotherapy | |
| | (n = 163) | | (n = 163) | | (n = 162) | |
| ORR*^{†}, n (%) | 58 | (35.6) | 56 | (34.4) | 61 | (37.7) |
| Estimated odds ratio vs. | 0.91 | (0.58-1.44) | 0.87 | (0.55-1.36) | | |
| chemotherapy (95% CI)^{‡} | | | | | | |
| Best objective response, n | | | | | | |
| (%) | | | | | | |
| Complete response^{†} | 1 | (0.6) | 0 | | 0 | |
| Partial response^{†} | 57 | (35.0) | 56 | (34.4) | 61 | (37.7) |
| Stable disease ≥ 6 weeks | 50 | (30.7) | 45 | (27.6) | 66 | (40.7) |
| Progressive disease | 53 | (32.5) | 59 | (36.2) | 25 | (15.4) |
| Not evaluable | 2 | (1.2) | 3 | (1.8) | 10 | (6.2) |

| | | | | |
|---|---|---|---|---|
| Median DOR^{§}, months | NR (9.7-NR) | NR (6.7-NR) | 4.4 | (3.5-5.5) |

| | | | |
|---|---|---|---|
| (95% CI) | | | |
| Remaining in response | | | |
| (%) at: | | | |
| 6 months | 66.9 | 67.6 | 32.4 |
| 12 months | 61.3 | 54.9 | 18.0 |

Primary analysis population.
*ORR by blinded inc ependent central review per RECIST vl.l is defined as the number (%) of patients with at least 1 visit response of complete response or partial response.
^{†}Responses included unconfirmed 1 responses.
^{‡}Analysis was performed using logistic regression adjusting for histology (squamc us vs. all other), with 95% CI calculated by profile likelihood. An odds ratio >1 favors immun therapy.
^{§}DOR was calculated using the Kaplan-Meier technique and was defined as the tii lie from the first documentation of complete response/partial response until the date of progress ion, death or the last evaluable RECIST assessment for patients that do not progress or for atients who progress or die after two or more missed visits. CI, confidence interval; DOR, durati on of response; NR, not reached; ORR, objective response rate; PD-L1, programmed cell c ieath ligand-1; PFS, progression-free survival; TC, tumor cell.

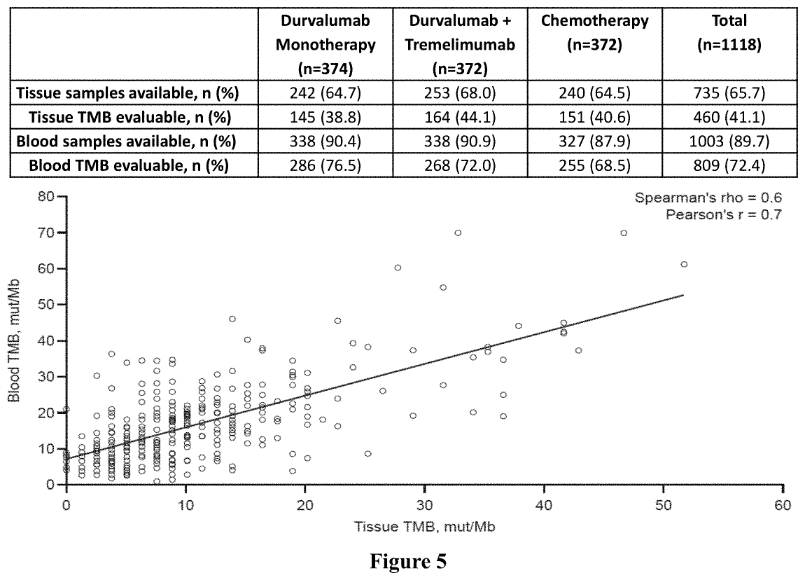

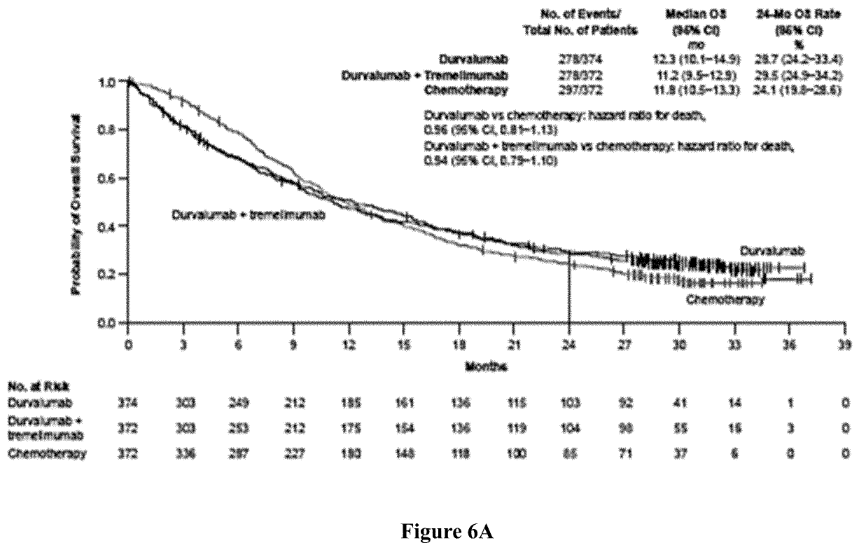

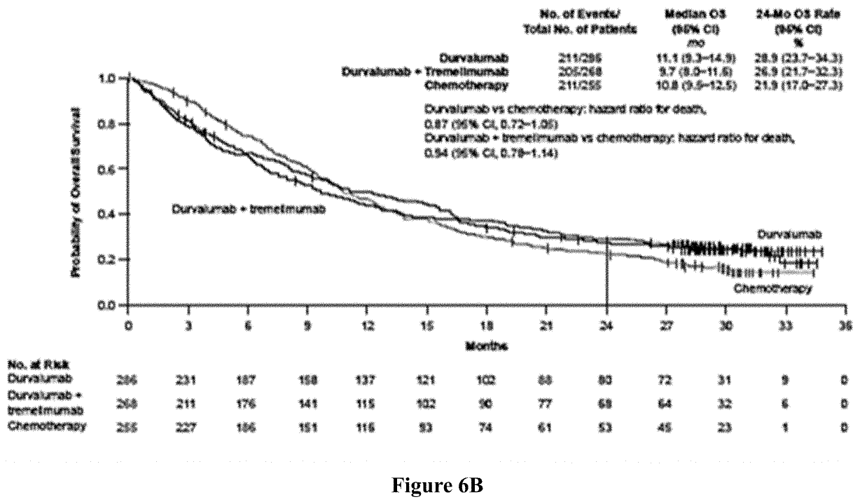

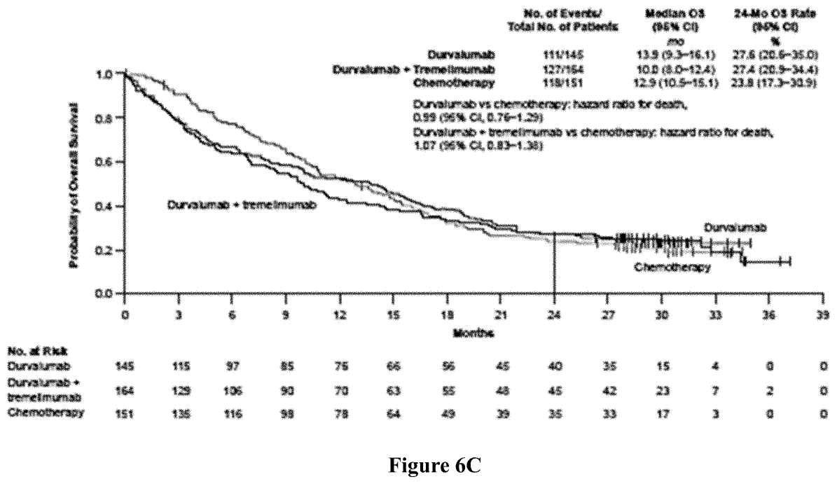

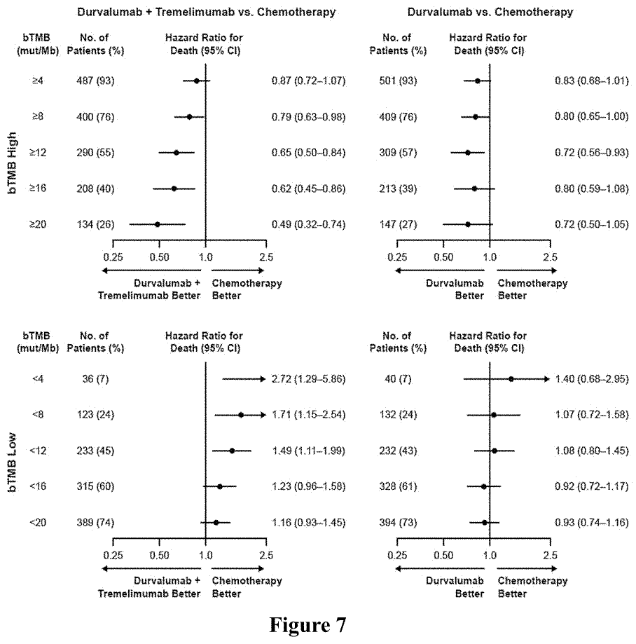

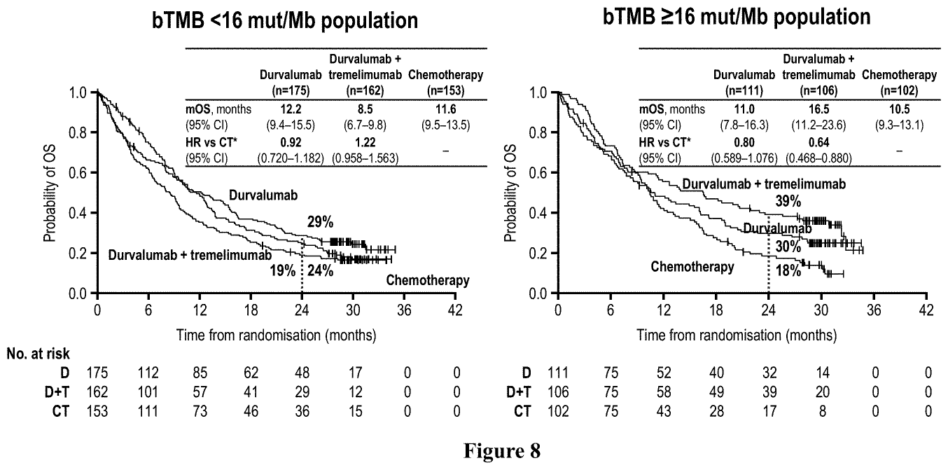

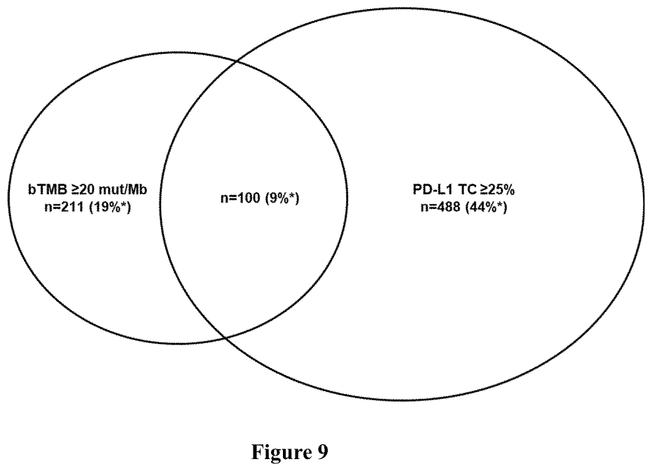

Blood and tissue pretreatment samples from 809 (72%) and 460 (41%) of 1118 randomized patients, respectively, were evaluable for TMB. TMB values did not correlate with PD-L1 expression levels (blood: Spearman's rho=0.05, Pearson's r=0.01; tissue: Spearman's rho=0.09, Pearson's r=0.06). Among patients with matched samples (n=352; 31% of randomized patients), bTMB and tTMB were correlated (Spearman's rho=0.6, Pearson's r=0.7; FIG. 5). Baseline characteristics in the bTMB and tTMB evaluable populations were consistent with the ITT population and balanced between treatment groups. OS in the TMB evaluable populations was consistent with the ITT population in the three treatment arms (FIGS. 6A-6C). HR for death improved gradually as the bTMB threshold was increased for durvalumab plus tremelimumab versus chemotherapy (FIGS. 7-8). Blood TMB ≥20 mut/Mb was selected for further analysis based on a clinically relevant effect size for durvalumab plus tremelimumab and the patient population deriving benefit. For context, tTMB ≥10 mut/Mb was studied based on a threshold shown to be predictive for PFS and response in previous trials of nivolumab plus ipilimumab in NSCLC (Hellmann et al., N. Engl. J. Med. 378(22): 2093-104 (2018); Ready et al., J. Clin. Oncol. 37(12): 992-1000 (2019)). Further analyses at tTMB thresholds above 10 mut/Mb were limited by small sample sizes. In patients with bTMB ≥20 mut/Mb or tTMB ≥10, there were greater proportions of patients with a smoking history and squamous histology compared with the corresponding lower TMB subgroups. Overlap between the bTMB ≥20 mut/Mb population and the PD-L1 TC ≥25% population was minimal (9% of randomized patients; FIG. 9).

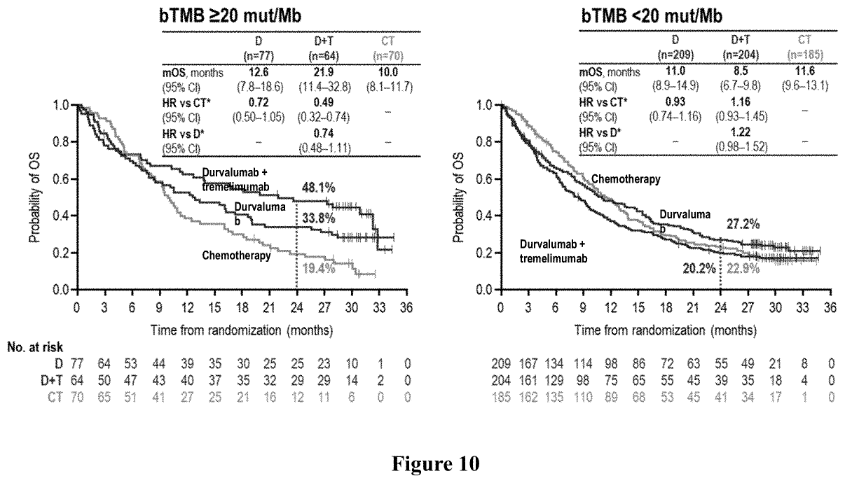

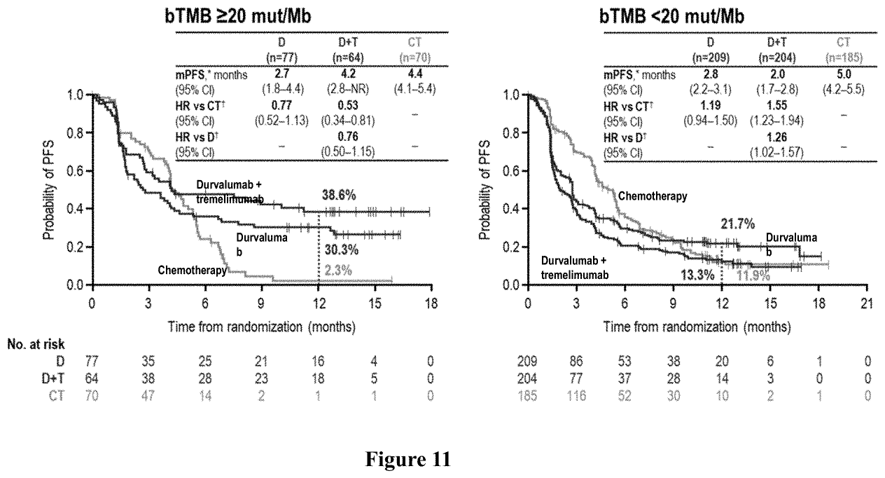

Blood TMB ≥20 mut/Mb was associated with improved OS for durvalumab plus tremelimumab versus chemotherapy (median, 21.9 vs. 10.0 months; unadjusted HR for death, 0.49 [95% CI, 0.32-0.74]; FIG. 10); 24-month OS rates were 48.1% (95% CI, 35.5-59.7) with durvalumab plus tremelimumab versus 19.4% (11.0-29.5) with chemotherapy. In contrast, there was no improvement in OS for durvalumab plus tremelimumab versus chemotherapy in patients with bTMB <20 mut/Mb (median, 8.5 vs. 11.6 months; unadjusted HR for death, 1.16 [95% CI, 0.93-1.45]; FIG. 10). Blood TMB ≥20 mut/Mb, but not bTMB <20 mut/Mb, was also associated with improved PFS (FIG. 11) and ORR (Table 3) for durvalumab plus tremelimumab versus chemotherapy.

TABLE 3
Analysis of Tumor Response among Patients with Blood TMB ≥20 mut/Mb and <20 mut/Mb.

| | Blood TMB ≥20 mut/Mb | | | Blood TMB <20 mut/Mb | | |
|---|---|---|---|---|---|---|
| | Durvalumab | Durvalumab + | | Durvalumab | Durvalumab + | |
| | Monotherapy | Tremelimumab | Chemotherapy | Monotherapy | Tremelimumab | Chemotherapy |
| | (n = 77) | (n = 64) | (n = 70) | (n = 209) | (n = 204) | (n = 185) |
| ORR*, n (%) | 23 | 31 | 15 | 43 | 34 | 58 |
| | (29.9) | (48.4) | (21.4) | (20.6) | (16.7) | (31.4) |
| Estimated odds | 1.56 | 3.44 | | 0.57 | 0.44 | |
| ratio | (0.74-3.36) | (1.65-7.46) | | (0.36-0.89) | (0.27-0.71) | |
| immunotherapy | | | | | | |
| vs. | | | | | | |
| chemotherapy | | | | | | |
| (95% CI)^{†} | | | | | | |
| Estimated odds | | 2.21 | | | 0.77 | |
| ratio | | (1.11-4.45) | | | (0.47-1.27) | |
| combination | | | | | | |
| therapy vs. | | | | | | |
| durvalumab | | | | | | |
| (95% CI)^{†} | | | | | | |
| Median DOR^{‡}, | NR | NR | 4.1 | NR | 11.1 | 4.1 |
| months (95% CI) | (NR-NR) | (NR-NR) | (3.0-4.3) | (5.9-NR) | (5.6-NR) | (2.8-5.6) |
| Remaining in | | | | | | |
| response (%) at: | | | | | | |
| 6 months | 86.5 | 85.6 | 14.4 | 64.0 | 66.6 | 33.3 |
| 12 months | 80.3 | 81.7 | 7.2 | 59.1 | 48.2 | 14.3 |

Primary analysis population.
*ORR by blinded independent central review per RECIST v1.1 is defined as the number (%) of patients with at least 1 visit response of complete response or partial response. Responses included unconfirmed responses.
^{†}Analysis was performed using logistic regression, with 95% CI calculated by profile likelihood. An odds ratio >1 favors the first comparator listed.
^{‡}DOR was calculated using the Kaplan-Meier technique and was defined as the time from the first documentation of complete response/partial response until the date of progression, death or the last evaluable RECIST assessment for patients that do not progress or for patients who progress or die after two or more missed visits.
CI, confidence interval; DOR, duration of response; Mb, megabase; mut, mutations; NR, not reached; ORR, objective response rate; PD-L1, programmed cell death ligand-1; TC, tumor cell; TMB, tumor mutational burden.

For patients with bTMB ≥20 mut/Mb receiving durvalumab alone, the median OS was 12.6 months (unadjusted HR for death vs. chemotherapy, 0.72; 95% CI, 0.50-1.05). The HR for death for durvalumab plus tremelimumab versus durvalumab was 0.74 (95% CI, 0.48-1.11; FIG. 10) supporting an additional contribution of tremelimumab.

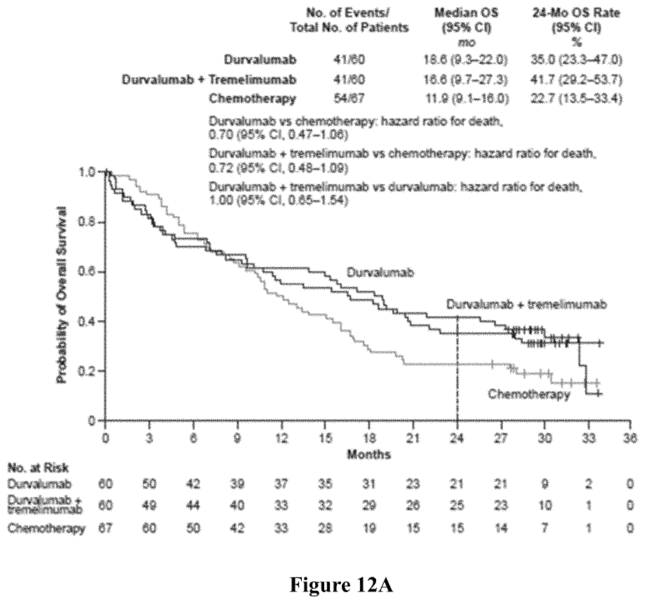

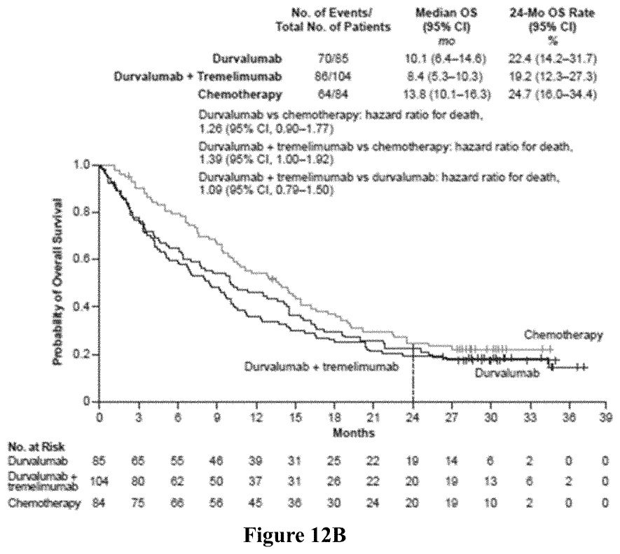

Tissue TMB ≥10 mut/Mb, but not tTMB <10 mut/Mb, was associated with numerically longer OS in both immunotherapy groups versus chemotherapy. The median OS was 16.6 months with durvalumab plus tremelimumab, 18.6 with durvalumab, and 11.9 with chemotherapy. The HR for death was 0.72 (95% CI, 0.48-1.09) for durvalumab plus tremelimumab versus chemotherapy and 0.70 (0.47-1.06) for durvalumab versus chemotherapy (FIGS. 12A-12B).

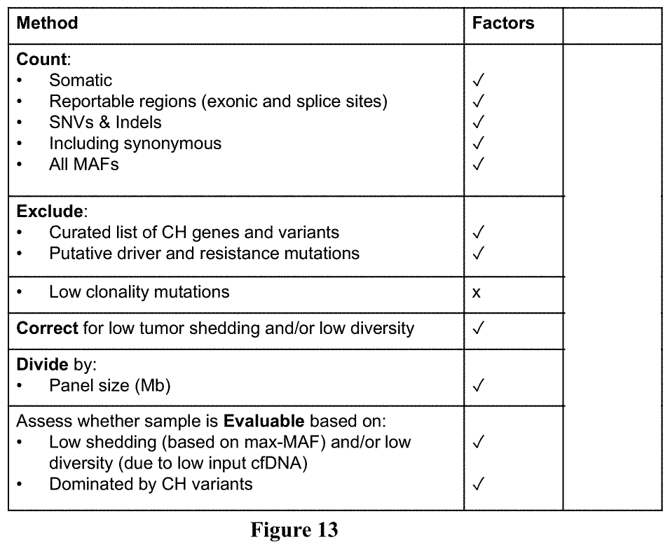

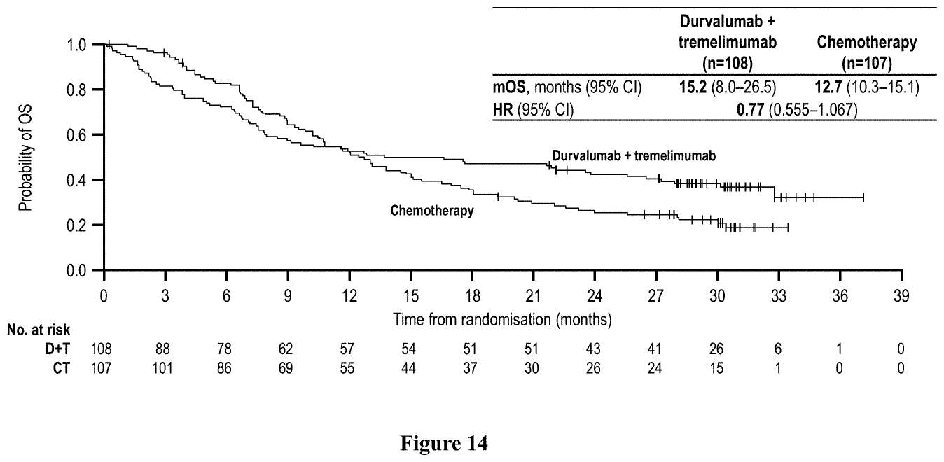

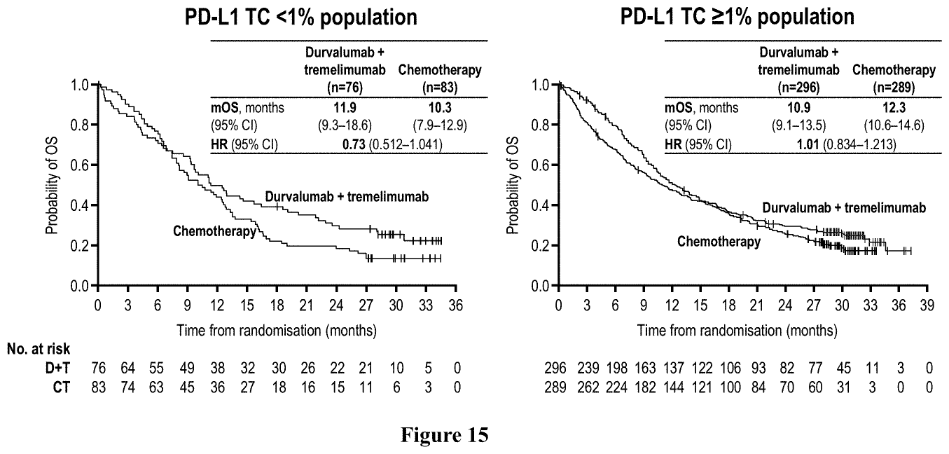

Two blood-based algorithms showed improvement of outcomes for D+T as compared to chemotherapy (V2 and V3; see FIG. 13). The V2 algorithm was selected for its simplicity over V3, though both showed similar predictive potential.

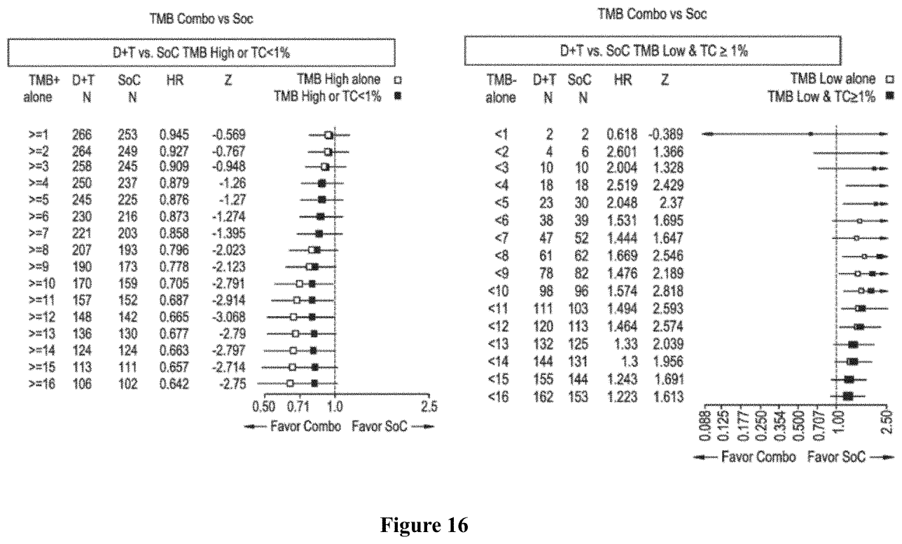

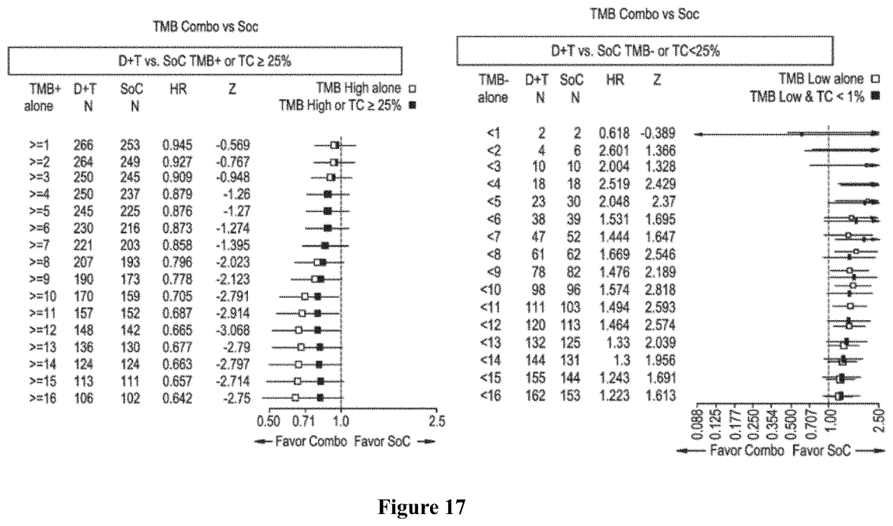

TMB was more predictive of OS than level of PD-L1 expression for D+T in MYSTIC regardless of the cut point used. This did not correlate with high PD-L1 expression, and thus, unique patient subsets were identified with bTMB (FIG. 9). Additionally, the combination of bTMB and PD-L1 expression increases the patient prevalence but reduces the effect size (FIGS. 16 and 17).

### 2. Safety

The median actual duration of treatment was 16.0 weeks (range, 0.4-148.6) for durvalumab; 16.0 (0.6-161.3) and 12.0 weeks (0.6-32.0) for durvalumab and tremelimumab, respectively, in the combination arm; and 17.9 weeks (1.1-137.4) for chemotherapy.

All-grade AEs that were considered by the investigator to be treatment-related (TRAEs) occurred in 54.2%, 60.1%, and 83.0% of patients treated with durvalumab, durvalumab plus tremelimumab, and chemotherapy, respectively. Rates of grade ≥3 TRAEs were lower with durvalumab (14.9%) and durvalumab plus tremelimumab (22.9%) than with chemotherapy (33.8%), and fewer patients had TRAEs leading to discontinuation in the durvalumab group (5.4% vs. 13.2% and 9.4%, respectively). Treatment-related deaths occurred in 2 patients (0.5%) in the durvalumab group, 6 (1.6%) in the durvalumab plus tremelimumab group, and 3 (0.9%) in the chemotherapy group. Safety in the PD-L1 TC ≥25% primary analysis population and the bTMB ≥20 mut/Mb population was consistent with the overall safety population.

Immune-mediated AEs were reported in 13.6% of patients in the durvalumab group, 28.3% in the durvalumab plus tremelimumab group, and 3.4% in the chemotherapy group. These events were of grade 3 or 4 in 4.1%, 10.8%, and 0.6% of patients, respectively.

Analyses identified a bTMB threshold of ≥20 mut/Mb for optimal OS benefit and clinically meaningful improvement in both PFS and response with durvalumab plus tremelimumab in this study.

### Example 2

### Mutations in ctDNA Associated with Sensitivity or Resistance to Immunotherapy in mNSCLC: Analysis from the MYSTIC Trial

This example investigated associations between selected mutations and survival outcomes. Circulating tumour DNA from baseline plasma specimens was profiled using the GuardantOMNI platform. Samples were available from 1003 patients (89.7% of ITT population). 943 samples were mutation-evaluable. Survival outcomes were analysed in patients with or without non-synonymous somatic mutations in STK11, KEAP1, or ARID1A, or KRAS.

The study demonstrated that poorer outcomes were observed across treatment arms in patients with metastatic NSCLC (“mNSCLC”) and mutations in serine/threonine kinase 11 gene (STK11) or Kelch-like ECH-associated protein 1 gene (KEAP1) as compared to those without the corresponding mutations. In patients receiving D+T, AT-rich interactive domain-containing protein 1A gene mutation (ARID1Am) was associated with survival benefits as compared with AT-rich interactive domain-containing protein 1A wild type gene (ARID1wt).

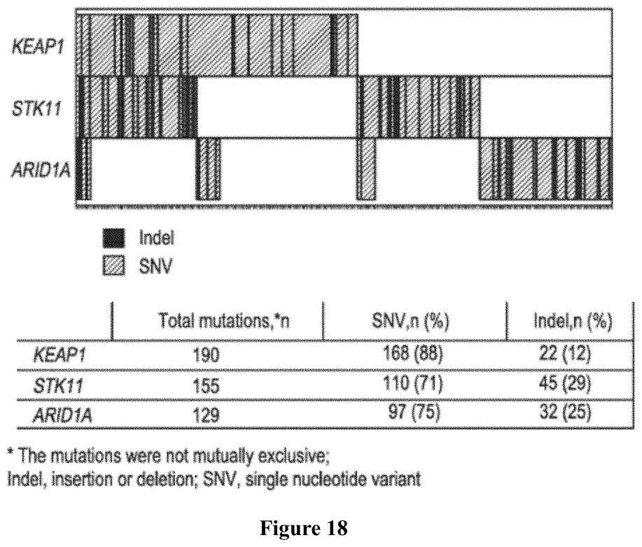

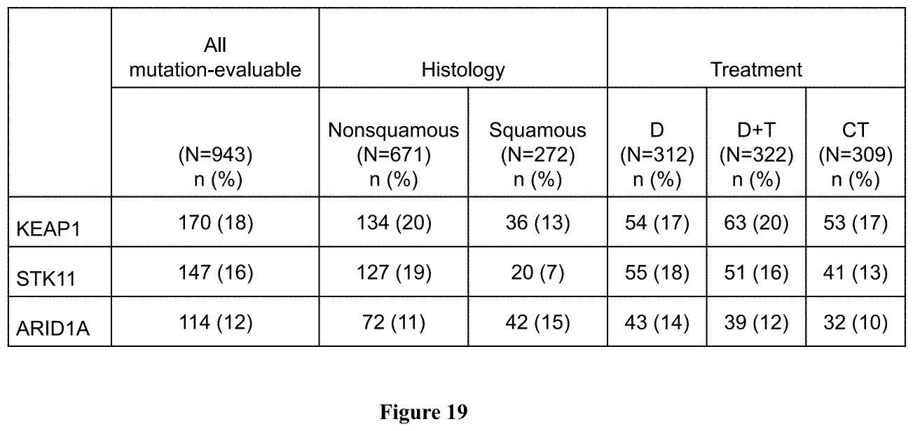

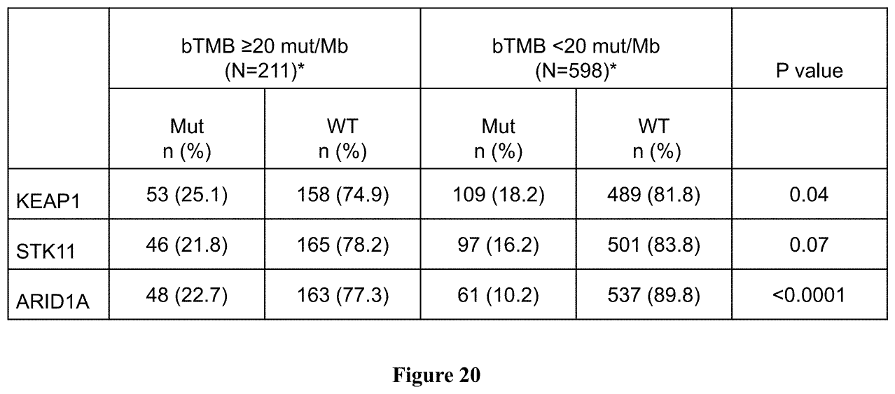

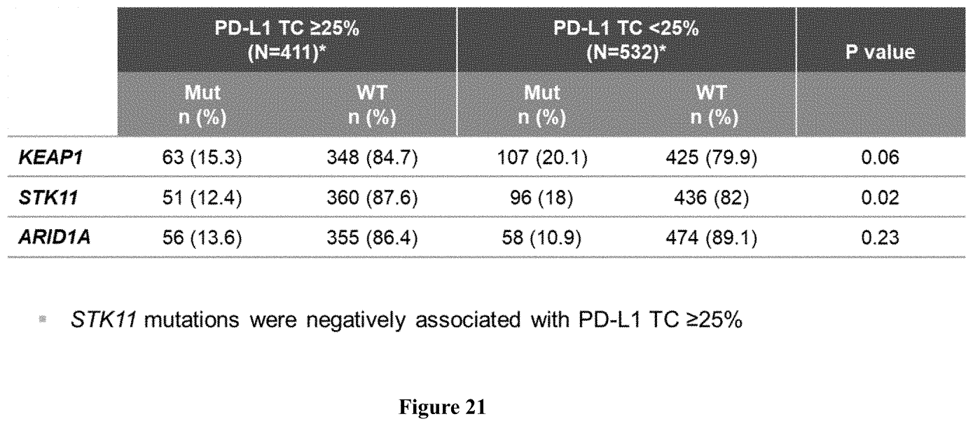

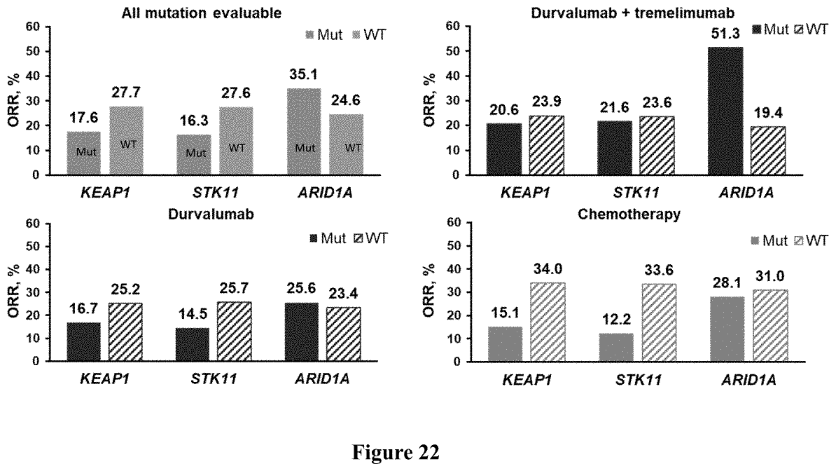

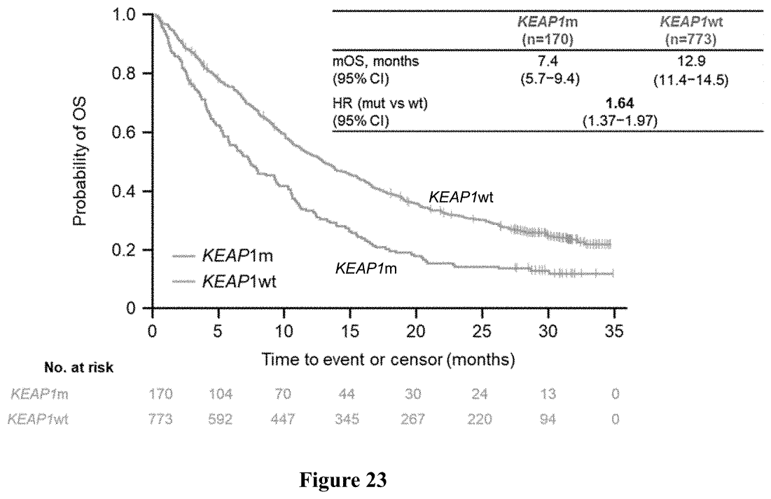

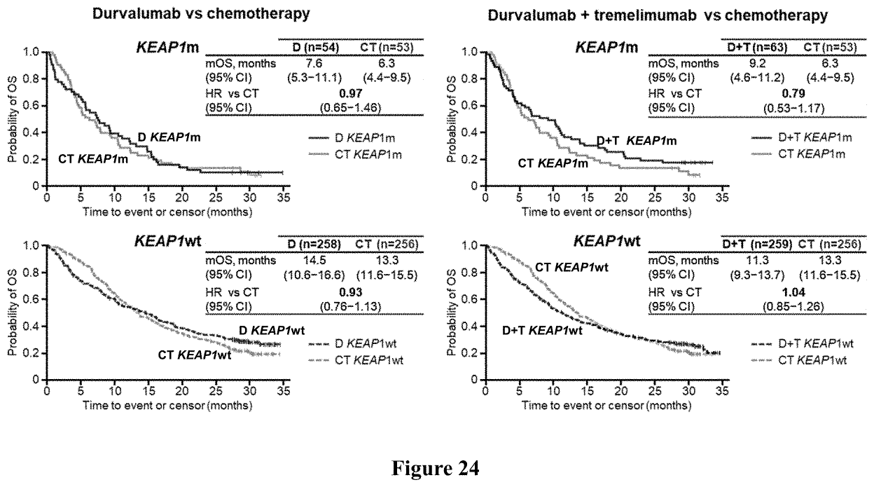

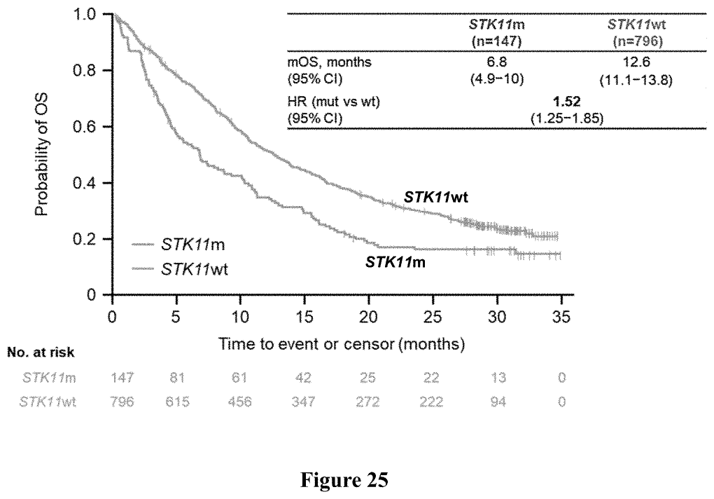

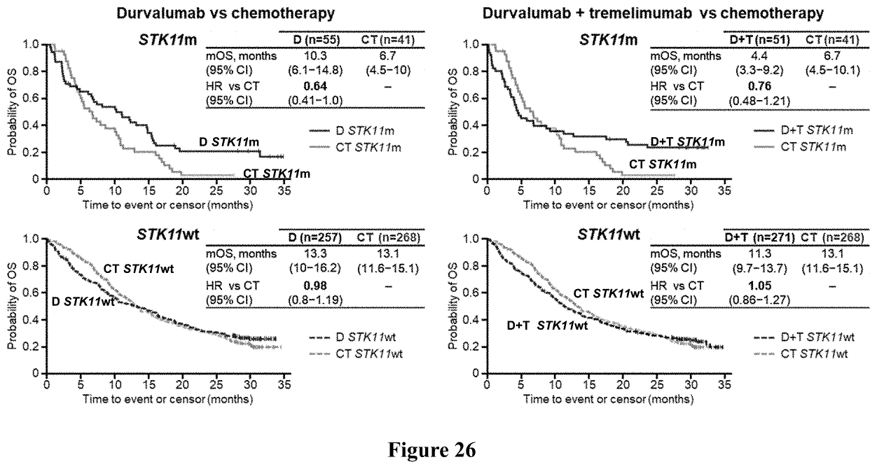

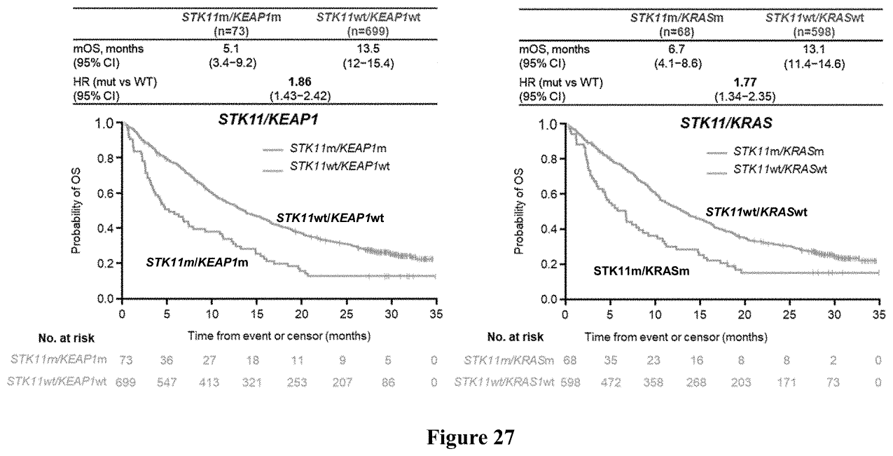

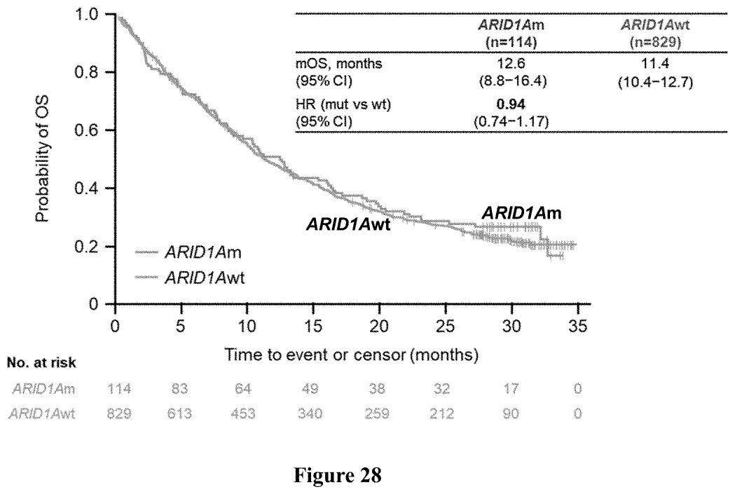

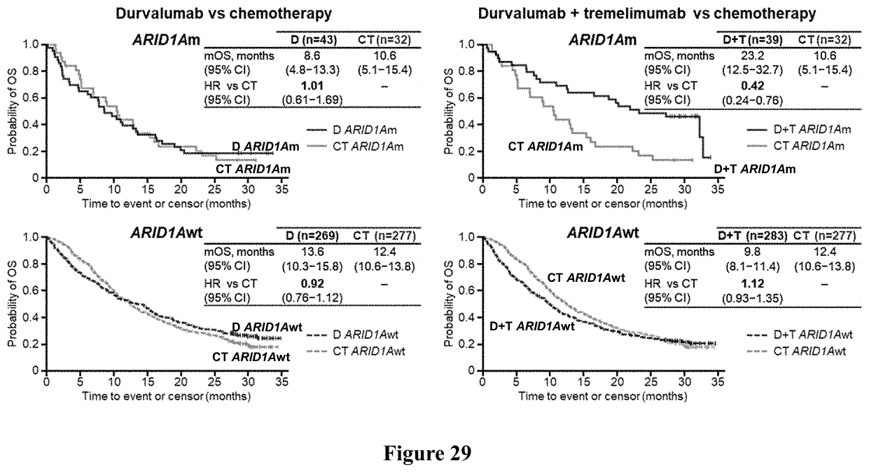

In the mutation-evaluable population, STK11m, KEAP1m, and ARID1Am frequencies were 16%, 18%, and 12%, respectively (19%, 20%, and 11% [nonsquamous]; 7%, 13%, and 15% [squamous]) (FIGS. 18-21). Across treatment arms, patients with STK11m or KEAP1m had a shorter median OS (“mOS”) than patients with STK11wt (D, 10.3 vs 13.3 months; D+T, 4.4 vs 11.3 months; CT, 6.7 vs 13.1 months) or KEAP1wt (D, 7.6 vs 14.6 months; D+T, 9.2 vs 11.3 months; CT, 6.3 vs 13.3 months) mNSCLC (FIGS. 22-27). In the D+T arm, patients with ARID1Am had a longer mOS than patients with ARID1Awt mNSCLC (D, 8.6 vs 13.7 months; D+T, 23.2 vs 9.8 months; CT, 10.6 vs 12.4 months) (FIGS. 28-29).

All patents and publications mentioned in this specification are herein incorporated by reference to the same extent as if each independent patent and publication was specifically and individually indicated to be incorporated by reference. Citation or identification of any reference in any section of this application shall not be construed as an admission that such reference is available as prior art to the present invention.

1. A method of treating non-small cell lung cancer (NSCLC) in a patient having a tumor mutational burden (TMB) that is ≥12 mutations/megabase, comprising administering durvalumab and tremelimumab to the patient.

2. The method of claim 1, wherein the TMB is ≥16 mutations/megabase.

3. The method of claim 1, wherein the TMB is ≥20 mutations/megabase.

4. The method of claim 1, wherein the method comprises administering 20 mg/kg of durvalumab.

5. The method of claim 1, wherein the method comprises administering 1 mg/kg of tremelimumab.

6. The method of claim 1, wherein the patient has not previously received systemic treatment for advanced or metastatic NSCLC.

7. The method of claim 1, wherein the patient has an overall survival of at least 22 months.

8. The method of claim 1 further comprising administering to the patient a chemotherapy agent.

9. The method of claim 8, wherein the chemotherapy agent comprises at least one of abraxane, carboplatin, gemcitabine, cisplatin, pemetrexed, or paclitaxel.

10. The method of claim 1, wherein the patient has a somatic mutation in at least one of serine/threonine kinase 11 gene (STK11), Kelch-like ECH-associated protein 1 gene (KEAP1), AT-rich interactive domain-containing protein 1A gene (ARID1A), or K-Ras gene.

11. A method of predicting success of a cancer treatment in a patient in need thereof, comprising determining the patient's tumor mutational burden (TMB), wherein a TMB that is ≥12 mutations/megabase predicts success of the treatment.

12. The method of claim 11, wherein a TMB that is ≥16 mutations/megabase predicts success of the treatment.

13. The method of claim 11, wherein a TMB that is ≥20 mutations/megabase predicts success of the treatment.

14. The method of claim 11, wherein the cancer treatment comprises treatment with durvalumab.

15. The method of claim 14, wherein the cancer treatment further comprises treatment with tremelimumab.

16. The method of claim 15, wherein the cancer treatment further comprises treatment with a chemotherapy agent.

17. The method of claim 16, wherein the chemotherapy agent comprises at least one of abraxane, carboplatin, gemcitabine, cisplatin, pemetrexed, or paclitaxel.

18. The method of claim 11, wherein the patient has a somatic mutation in at least one of serine/threonine kinase 11 gene (STK11), Kelch-like ECH-associated protein 1 gene (KEAP1), AT-rich interactive domain-containing protein 1A gene (ARID1A), or K-Ras gene.
